(19) United States
(12) Patent Application Publication
(10) Pub. No.: US 2008/0312859 A1
(43) Pub. Date: Dec. 18, 2008

(54) Method of Calibrating a System for Measuring the Concentration of Substances in Body and an Apparatus for Exercising the Method

(75) Inventors: Ole Skyggebjerg, Hillerod (DK); Arne Stjernholm Madsen, Valby (DK); Michael Gerstenberg, Horsholm (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO NORDISK A/S, Bagsvaerd (DK)

(21) Appl. No.: 11/661,866

(22) PCT Filed: Sep. 5, 2005
(86) PCT No.: PCT/EP05/54359

§ 371 Date: Aug. 25, 2008

(30) Foreign Application Priority Data
Sep. 3, 2004 (DK) PA 2004 01335

(51) Int. Cl. G06F 19/00; G01N 33/48

(52) U.S. Cl. 702/85; 702/19

(57) ABSTRACT

This invention relates to procedures for the calibration of systems for continuously measuring the concentration of substances in a body fluid. The system comprises first and second sensors adapted for subcutaneous insertion and an electronic calculator unit adapted for measuring signals from the two sensors. The system is calibrated following the steps of: a) introducing the first sensor subcutaneously, b) calibrating the first sensor, c) obtaining sensor data S₁(t) provided by the first sensor, d) introducing the second sensor subcutaneously, e) obtaining sensor data S₂(t) provided by the second sensor, f) determining the rate of change over time δR(t)/δt, R(t) being a signal which correlates to sensor data S₂(t) over time, and g) performing a calibration of the second sensor when δR(t)/δt is less than a predetermined value, said calibration of the second sensor being performed using sensor data S₁(t) obtained by the first sensor.

### FIELD OF THE INVENTION

This invention relates to calibration procedures for biosensors, in particular transcutaneous electrochemical sensors suitable for in vivo measurement of metabolites.

### BACKGROUND OF THE INVENTION

In recent years, a variety of implantable sensors have been developed for in vivo measurements of various biological parameters. Among these transcutaneous sensors (ie sensors mounted through the skin) show promise for real-time measuring of important biological parameters like acidity of the blood and concentration of metabolites and blood gasses.

One of the most prominent examples of the use of implantable sensors is within the field of blood glucose (BG) measurements. BG information is of the utmost importance to diabetics, as these readings are instrumental in the adjustment of the treatment regimen.

The conventional way to obtain BG information is by applying minute amounts of blood to test strips. Although simple and reliable, this method gives only discrete readings and thus not a complete understanding of the BG at any time. A new development is transcutaneous sensors where the sensor is implanted under the skin. As the sensor is always in contact with biological fluids, this opens the possibility for continuous measurements. Continuous BG readings obtained with little or no delay will be particularly useful in numerous ways. First of all, the continuous monitoring will help preventing hypoglycaemic incidents and thus contribute to a vast increase in the quality of life of the diabetic patient.

Although the invention described in this application is not limited to calibration of systems for BG measurements, BG measurements will be used in the following text to exemplify all relevant aspects of the invention.

In general, readings from a transcutaneous sensor reflect only to some extent the value found in undisturbed tissue. An exact reading is not obtainable due to the metabolic changes in the tissue caused by the damage inflicted during insertion. The relation between readings in disturbed tissue and the actual value in undisturbed tissue is therefore unknown in the general case.

If transcutaneous sensors are used to indicate the concentration of species in the bloodstream, the relation between the reading and the actual value becomes even more complex due to time lag between the concentration found in the blood and the value read by the sensor. This is the case in particular for BG measurements, as BG sensors are most often mounted in the subcutaneous tissue although the value of interest is the concentration of glucose present in the bloodstream.

To summarise, the measured value of eg glucose found in the subcutaneous tissue reflects to some degree the concentration found in the bloodstream although a time lag between the reading and the actual value exists. For glucose the time-corrected concentration in the subcutaneous tissue is in general lower than in the bloodstream due to physiological factors as well as tissue damage. Thus the readings even from an ideal subcutaneous sensor will represent only the actual value found in the blood if corrected for the unknown proportionality factor as well as time-lag.

In patent application No. US 2002/0161288 A1 an approach to calibration is claimed that employs numerous calibration values taken at predetermined intervals. According to the method described in this patent, sampling has to be carried out at predetermined intervals until two consecutive calibration factors fall within a certain interval. Thereafter a readout of the measured glucose concentration can be presented on a display.

If follows that the prior art is vitiated by the drawback that—when a new sensor is to be started up—it is necessary to perform calibrations and then wait a while for it to be verified, by means of electronic circuits, that the deviation between the measurements/calibrations is sufficiently low. It is a further considerable drawback that the user has to take out blood samples eg from a fingertip each time a calibration is to be performed; a procedure which is associated with much discomfort. It is a fact that the users associate this procedure with a substantially more pronounced sense of discomfort than is the case for the act of having to inject oneself to administer a dose of insulin.

EP patent application No. 314.027 describes a method for the simultaneous or alternating activation of two identical sensors for biological and physiological parameters on a common analysis and display unit. The alternating cycles of activating and inactivating the particular sensors described is due to the fact that these particular sensors are not able to work in a continuous mode. Thus, one of the sensors is activated as a measuring sensor in a measuring phase and another sensor as a standby sensor in a standby phase, i.e. the two sensors are driven sequentially. The two sensors are continuously subjected to the measurement site during a prolonged time period consisting of several measurement cycles, and in order for the sensors to provide acceptable measurements, each sensor is deactivated in turn while the other sensor is active. Although the system described in EP patent application No. 314.027 consists of a least two discrete sensors, these sensors are to be considered as a single sensor assembly allowing for continuous monitoring although the single sensors requires to be driven discontinuously.

### THE OBJECT OF THE INVENTION

It is the object of the invention to provide a method of calibrating a subcutaneous sensor which recently has been inserted in subcutaneous tissue, the method providing simple and rapid calibration while requiring no or only a few reference calibrations.

This object is achieved in that the calibration of a newly inserted sensor is performed by means of signals from another sensor that was introduced subcutaneously for a period of time preceding the insertion of said new sensor. The signals which has been picked up by the two sensors are compared during initialisation of the new sensor, and by comparing the signals during this phase, a criterion for estimating a satisfactory correspondence between the two signals is established.

In this manner the new sensor is calibrated by means of the signals from the previously arranged sensor, and therefore the new sensor will very quickly produce results that are just as good as those of the previously arranged sensor.

Owing to operation due to changes in the tissue, the measurement accuracy in connection with the initially arranged sensor can be reduced with time, and therefore it is recommended to perform a reference calibration on a blood sample, eg by means of the well-known prior art strip technique.

Preferably a central electronic calculator circuit or electronic calculator unit is used and two transmitter/receiver circuits that are connected to each their sensor during the calibration period. The use of such sensors is well known, in particular in connection with such sensors that are connected to a respective transmitter/receiver circuit that preferably exchanges information wirelessly with the central electronic calculator circuit. By such systems it is common to use a disposable electrode that is connected to a multiple-use transmitter/receiver circuit which therefore has to be charged at intervals, whereby it is already known in the art to have to switch between two transmitter/receiver circuits. Thus it follows that the invention does not presuppose use of further components; rather it benefits neatly from the circumstance that it is common to use two different transmitter/receiver circuits that are, in accordance with the invention, used simultaneously during a calibration period to calibrate the new sensor by means of the old sensor.

Preferably the electronic circuit is configured for providing a message to the user as soon as there is sufficient correspondence between the signals from the two sensors, following which the user is able to remove the old sensor and continue to use the new one. The circuit can also be configured such that it encourages the user to perform a reference calibration measurement, eg in case problems occur in connection with the execution of the calibration principle according to the invention.

The signals from the two sensors can be compared in various ways. The comparison is relatively simple when there is no significant timelag between the sensor signals as will be the case when the sensors are arranged relatively close to each other. If it is desired to arrange the new sensor on the body relatively far from the old sensor, a timelag may occur between the signals; however, this is solved by the prior art known per se, such as cross-correlation analysis.

It is a major problem in the calibration to determine the time lag prevailing between a given time of a blood-glucose concentration measurement in blood and the time when a corresponding, delayed measurement in the body fluid can be performed. Thus, according to the invention it may be expedient to compare, during the signal processing, a number of mutually time-lagged versions of the signals from the new sensor to the signal from the old sensor

According to one embodiment the electronic calculator circuit can also be configured for calculating and displaying the uncertainty interval, i.e. the degree of accuracy of the measurement from the new sensor. It can be accomplished by means of the technique taught in the co-pending PCT application entitled “System and method for estimating the glucose concentration in blood” which is filed on the same date and by the same applicant as the present invention and which claims the priority of Danish patent application No PA 2004 01333.

The application also relates to an apparatus for subcutaneous measurement of the concentration of substances in body fluid; eg glucose. The apparatus is characterised in that the electronic calculator circuit is configured for calculating and displaying the uncertainty interval of the measurement from the sensor. Preferably each sensor comprises a respective multiple-use electronic transmitter circuit, which is not unknown, see above; however by using the sensors simultaneously during a calibration period and calibrating the new sensor in accordance with the old one, an entirely unique improvement of the prior art is accomplished by very simple means.

Preferably the central calculator unit is configured for receiving reference calibration signals that can be received wirelessly from a measurement apparatus for measuring the blood glucose concentration in a blood sample; however, it is also an option that such measuring device can be built integrally with the apparatus according to the invention. Moreover, the apparatus can be configured for calculating an uncertainty interval of the glucose concentration measurement and displaying that interval on a display. Preferably the uncertainty interval is displayed with a graphical representation due to so many diabetics being visually impaired.

In a further aspect of the invention, the system is calibrated following the steps of: a) introducing a first sensor subcutaneously, b) calibrating the first sensor, c) obtaining sensor data S₁(t) provided by the first sensor, d) introducing a second sensor subcutaneously, e) obtaining sensor data S₂(t) provided by the second sensor, f) determining the rate of change over time δR(t)/δt, R(t) being a signal which correlates to sensor data S₂(t) over time, and g) performing a calibration of the second sensor when δR(t)/δt is less than a predetermined value, said calibration of the second sensor being performed using sensor data S₁(t) obtained by the first sensor.

The invention will now be explained in further detail with reference to the following description of exemplary embodiments, reference being made to the drawing, in which:

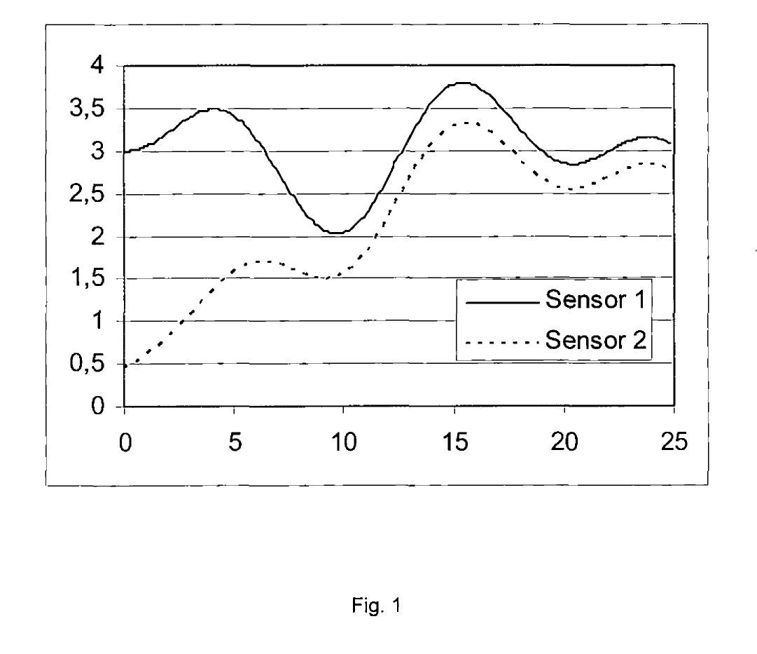

FIG. 1 shows the measurement signals from an old and a new sensor;

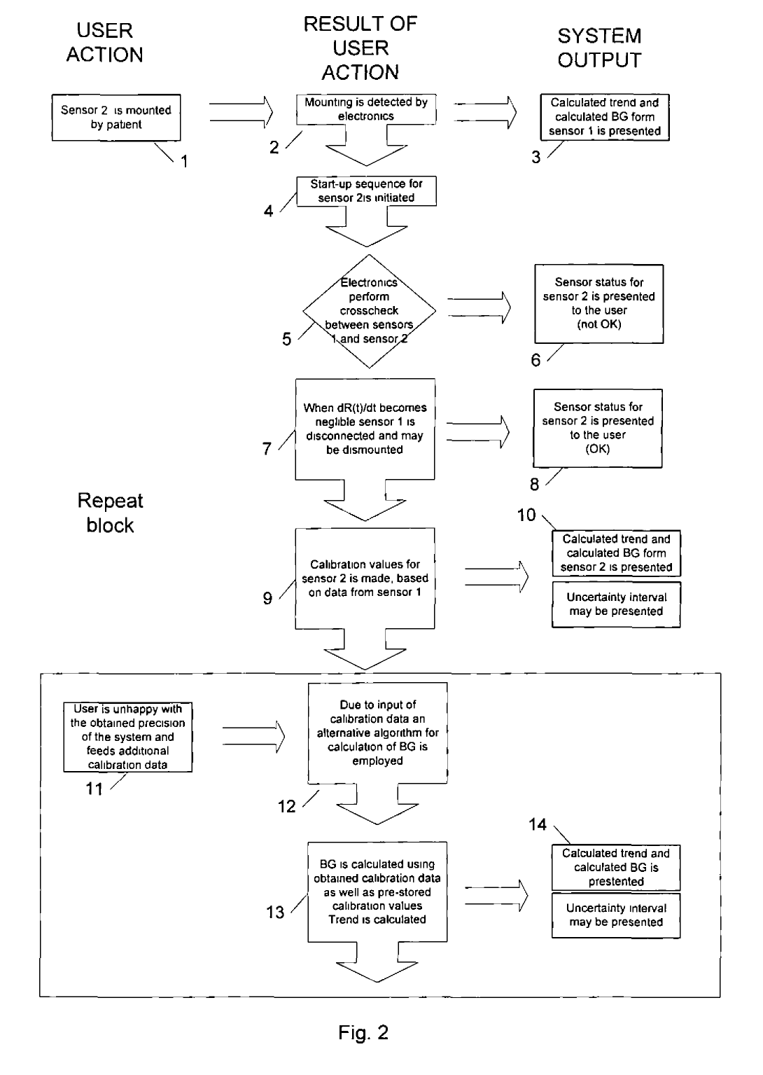

FIG. 2 shows a flow chart of an example of a calculation process with a view to determining when there is sufficient correspondence between the signals of FIG. 1; while

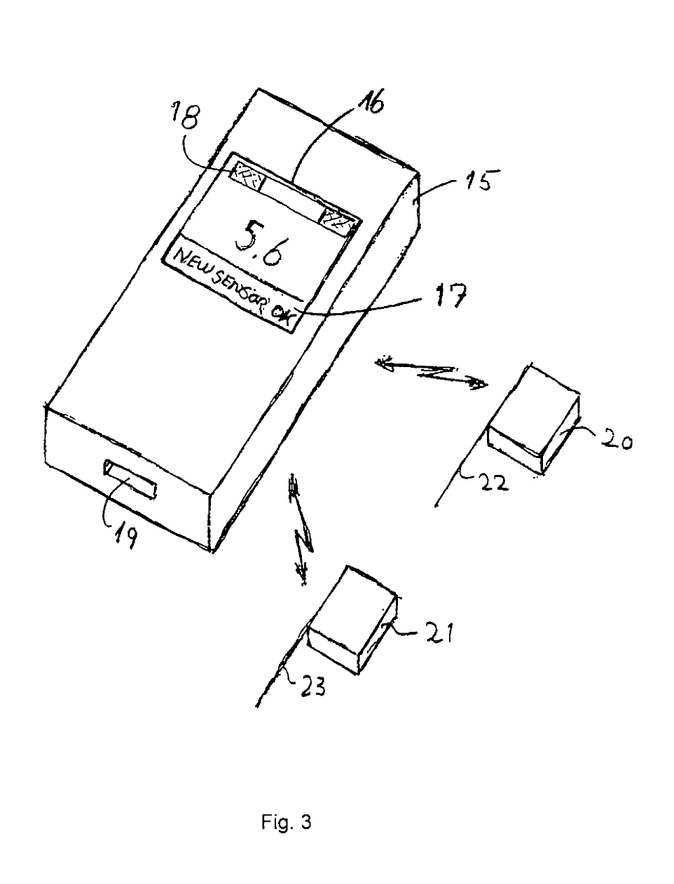

FIG. 3 shows an exemplary apparatus for exercising the method according to the invention.

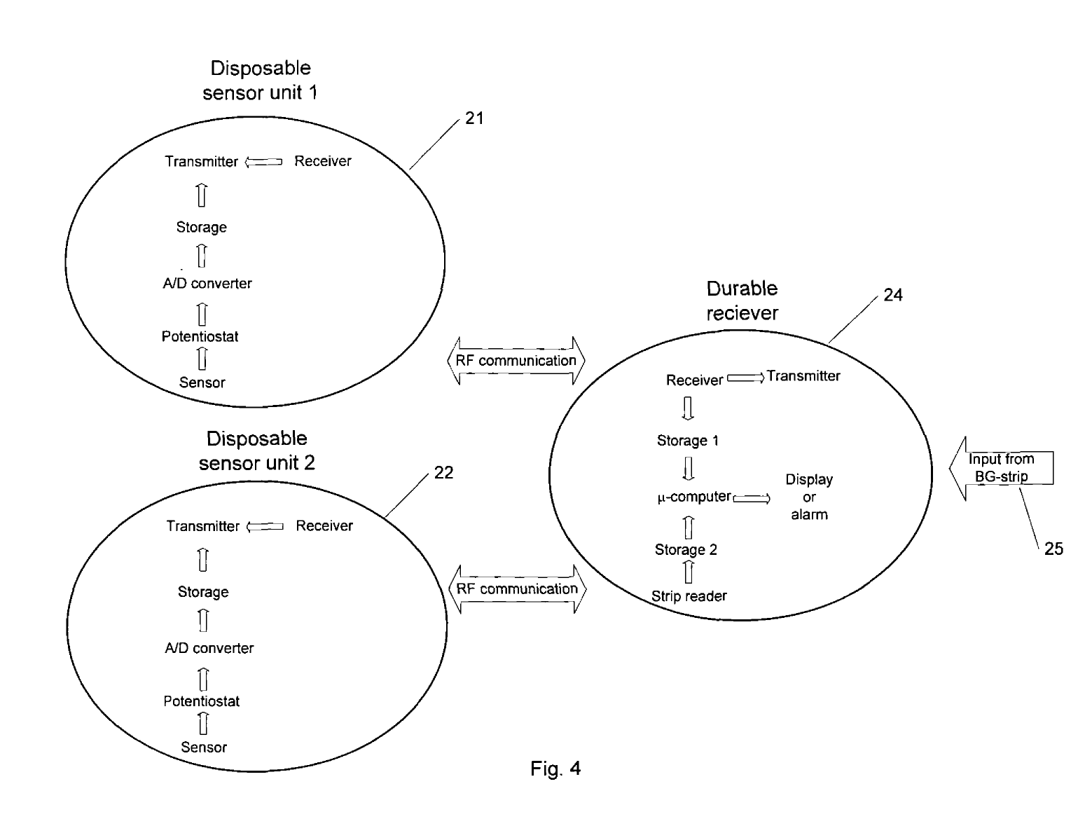

FIG. 4 illustrates the electronic functionality units that may partake in the apparatus, eg the one shown in FIG. 3.

### DETAILED PART OF THE DESCRIPTION

FIG. 1 shows sensor signals from a previously implanted sensor 1 and a sensor 2 which has just been implanted.

Sensor 1 is working during the whole time interval. At the time t=0 sensor 2 is mounted, Full correct signal is at time t=0 not received from sensor 1. This is first achieved at time=20.

Multiple methods may be employed to correlate the two sensor signals to each other.

According to one embodiment of the of the invention the ratio of the signal from the two sensors relative to each other is measured as

$$R ( t ) = \frac{{S}_{2} ( t )}{{S}_{1} ( t )}$$

### Where

S₁(t) is the signal from sensor 1 and S₂(t) is the signal from sensor 2

Sensor 1 and sensor 2 are carried simultaneously until the criteria

$$\frac{δ R ( t )}{δ t} \leq ɛ$$

i.e. the ratio of the signals from sensor 1 and sensor 2 are constant. This situation is achieved approx. at time=20 in the figure.

At time=20 the value of BG read from sensor 1 can directly be used for calibration of sensor 2.

If a calibration using a strip measurement is carried out in the time-interval t=0 . . . t=20 this calibration applies to the signal from sensor 1. If a calibration is carried out after t=20 this strip calibration will be used to correct the measurements obtained using sensor 2.

By analyzing R(t) it will be possible to detect whether sensor 2 is functioning properly.

If e.g. the condition

$$\frac{δ R ( t )}{δ t} \leq ɛ$$

is achieved too fast or too slowly this might indicate that sensor 2 is not properly mounted. The condition above is typically reached within 1-2 hours.

If the ratio R(t) is not within certain limits it is an indication that either sensor 1 or sensor 2 is malfunctioning.

FIG. 2 shows a flowchart illustrating how a user can exercise the method according to the invention, wherein sensor 1 refers to a sensor that has been arranged in the tissue for some time, wherein the sensor has emitted measurement signals based on some adequate kind of calibration. Sensor 2 refers to a new sensor arranged by the user with a view to enable replacement of sensor 1 due to the fact that, over time, such sensor has to be changed.

By 1 it is shown that the sensor is arranged by the user. Preferably sensor 2 is arranged in the vicinity of sensor 7, which provides the advantage that the signals of the sensors can readily be compared without any significant time-lag in relation to each other. However, the invention also relates to the situation where sensor 2 is arranged so far away from sensor 1 that a time-lag may occur between the signals, a phenomenon that can easily be compensated for by supplementing the above-referenced comparative processes with cross-correlation analysis, frequency analysis or other technique known per se.

The electronic circuits in the central calculator unit performs, as shown in function 2, a control of sensor 2, and according to the invention the central calculator unit is configured for being able to operate both with sensor 1 and sensor 2 to the effect that the results from sensor 1 can be calculated and displayed as shown in function 3 simultaneously with sensor 2 being active. In function 4 various further start-up procedures are performed, following which the signals from sensor 1 and sensor 2 are compared in function 5. According to the invention, for instance function 6 provides a clear indication to the user when sensor 2 can be taken into use. In function 6 it is shown that sensor 2 cannot be taken into use yet, as it is not until in function 7 it is detected that the error is sufficiently small, following which the user is informed to that effect in function 8.

Then sensor 1 can be discarded and all subsequent calculations and displays occur exclusively on the basis of sensor 2 as shown by the functions 9 and 10.

The accuracy of the measured glucose concentration depends on how long it has been since a reference calibration measurement was performed, ie since the glucose concentration in the blood was last examined, eg by means of a strip test measurement, see our comments above regarding strip measurement in the time interval t=0 . . . t=20.

However, it will also be possible in practice to perform further reference calibration measurements if the user is not satisfied with the accuracy of the system, see function 11 in FIG. 2. Functions 11-14 can be performed repeatedly in response to the needs of the user, and/or the apparatus is configured for displaying the interval within which the measurement is comprised. (Further details regarding the understanding of that calculation, please refer to Danish patent application No. . . . filed on the same date as the present application and by the same applicant.) In this manner it is possible to accomplish a very accurate calibration of sensor 2; however, it is noted that the forte of the invention relies entirely on the novel technical effect that sensor 2 can be used for reliable measurements very shortly after positioning of sensor 2 due to sensor 1 being used for calibrating sensor 2.

FIG. 3 shows a portable central unit 15 being, according to the invention, configured for simultaneous communication with at least two sensors, preferably via wireless communication. Each of the sensors comprises an electrode 22 or 23 that is connected to an associated electronic circuit 20 or 21, respectively. Preferably the electronic circuits 20 and 21 are multiple-use circuits that are connected to new electrodes when the electrode's lifetime is over.

According to the invention, the central calculator unit 15 is configured for receiving signals from the two sensors simultaneously in a calibration phase, wherein the signals of the sensor arranged first are used to calibrate the signals of the sensor arranged later. Usually, outside the calibration periods communication will take place only with the one of the sensors, while the electronic circuit of the second sensor is eg being charged.

In accordance with the invention the unit 15 may feature a display comprising an indication whether the new sensor is calibrated correctly or not, see 17 in FIG. 3 and see functions 6 and 8 in FIG. 2. As soon as sensor 2 is calibrated, an indication to that effect will be made clearly available to the user who then removes sensor 1. By 19 is shown an opening for introducing a test strip for performing reference calibration measurements. Such reference measurements will be used on the sensor that is active, and if both sensors are active during a calibration period, the reference calibration will typically be used on the older of the sensors, the calculation circuits being configured for also taking into consideration the history of a sensor. The display 16 also features an area 18 configured to function as an indication of an interval of the uncertainty of the glucose concentration measurement. Further details of this function will appear from co-pending PCT application entitled “System and method for estimating the glucose concentration in blood” which is filed on the same date and by the same applicant as the present invention and which claims the priority of Danish patent application No PA 2004 01333. A combination of these latter features and the present invention will constitute an entirely extraordinary improvement of the performance of the new sensor; however, the techniques according to the two applications each separately constitutes a great improvement over the prior art.

FIG. 4 illustrates the typical circuit components that are needed in the apparatus to exercise the method according to the invention. The figure shows disposal sensor units 21 and 22, wherein the electrode as such is combined with the electronic circuits to form one single disposable unit. By means of the circuits shown in units 21 and 22, those functions can be performed that are necessary for being able to perform the sensor functions shown and explained in connection with FIG. 2. The functions that remain can be performed by means of the electronic circuits shown in the durable receiver 24. 25 designates input from the BG-strip, which may be accomplished either by a test-strip being introduced into the opening 19 of the apparatus 15 in FIG. 3, or by a separate BG-strip measurement device being provided; and that by information from that device being transferable to the durable receiver, preferably via wireless communication.

It will be understood that the circuits that are present in units 21, 22 and 24 can also be configured for performing other signal processing functions known per se, such as utilisation of history for the sensors used, receipt of particular calibration information from the sensors, further sophisticated and known mathematical analyses known per se with a view to improving either the measurement results and/or the options of predicting the uncertainty of the calculations, see the above-referenced parallel application.

1. A method of initial calibration of a newly mounted sensor for continuous measuring the concentration of substances in body fluid, e.g. the glucose concentration, the system comprising an already mounted and calibrated first subcutaneous sensor, a newly mounted uncalibrated second subcutaneous sensor and an electronic calculator unit adapted for measuring signals from said sensors, said signals being measured over time, said method comprising the steps of:
obtaining sensor data S₁(t) provided by the first sensor,
obtaining sensor data S₂(t) provided by the second sensor,
determining the rate of change over time δR(t)/δt, R(t) being a signal which correlates to sensor data S₂(t) over time,
performing a calibration of the second sensor when δR(t)/δt is less than a predetermined value, said calibration of the second sensor being performed using sensor data S₁ (t) obtained by the first sensor.

2. The method as defined in claim 1, wherein R(t) is determined by
$$R ( t ) = \frac{{S}_{2} ( t )}{{S}_{1} ( t )}$$

3. The method as defined in claim 1, wherein a reference calibration measurement of the body fluid concentration is performed; and wherein the result is transmitted to the electronic calculator unit.

4. The method as defined in any of claim 1, wherein the electronic calculator unit comprises two transmitter/receiver circuits which are coupled to each their sensor from the time of introducing the second sensor until δR(t)/δt becomes less than a predetermined value.

5. The method as defined in claim 4, wherein the electronic calculator unit transmits a message to the user when there is sufficient correspondence between the signals from the two sensors.

6. The method as defined in any of claim 3, wherein the electronic calculator unit transmits a message to the user to perform a reference calibration measurement.

7. The method as defined in any of claim 1, wherein a cross-correlation analysis is performed on the signals from the two sensors.

8. The method as defined in claim 7, wherein curves are recorded representing the signals from the two sensors; and that the respective areas between the curves, measured during predetermined respective periods of time, are compared to each other.

9. The method as defined in any of claim 7, wherein the signals from the second sensor are divided into a number of signals that are mutually time-lagged; and that each of the time-lagged signals are compared to the signals from the first sensor.

10. The method as defined in any of claim 1, wherein the electronic calculator unit is configured for calculating and displaying the uncertainty interval of the measurement from the sensor.

11. An apparatus for subcutaneous measurement of the concentration of substances in body fluid; e.g. the glucose concentration, said apparatus being adapted for receiving signals from a first and at least a second sensor, said signals being measured over time, the apparatus having means for obtaining sensor data S₁ (t) obtained by the first sensor and means for obtaining sensor data S₂(t) provided by the second sensor, the apparatus further comprising:
means for determining the rate of change over time δR(t)/δt, R(t) being a signal which correlates to sensor data S₂(t) over time,
means for evaluating when δR(t)/δt is less than a predetermined value, indicating that a valid calibration of the second sensor can be carried out.

12. The apparatus as defined in claim 11, wherein the apparatus has means for calibrating the second sensor using sensor data S₁ (t) obtained by the first sensor.

13. The apparatus as defined in claim 11, wherein the apparatus has means for signalling when δR(t)/δt is less than a predetermined value.

14. The apparatus as defined in any of claim 11, wherein R(t) is determined by
$$R ( t ) = \frac{{S}_{2} ( t )}{{S}_{1} ( t )}$$

15. The apparatus as defined in any of claim 11, wherein the apparatus is configured for simultaneously receiving, during a calibration period, measurement signals from the two subcutaneous sensors; and performing sensor calibration by comparison of the signals received from the two sensors.

16. The apparatus as defined in any of claim 11, wherein the apparatus is configured for receiving reference calibration measurements.

17. The apparatus as defined in claim 16, wherein the apparatus comprises a measuring device for measuring the blood-glucose concentration in a blood sample.

18. The apparatus as defined in any of claim 11, wherein the apparatus is configured for calculating and displaying the uncertainty interval of the measurement from the first and/or the second sensor; and that the apparatus comprises a display configured for displaying the uncertainty interval.

19. The apparatus as defined in claim 18, wherein the display is configured for graphical representation of the uncertainty interval.
